(19) United States
(12) Patent Application Publication
(10) Pub. No.: US 2026/0262933 A1
(43) Pub. Date: Sep. 10, 2026

(54) MEDICAL APPARATUS FOR MULTI-ANGLE IMAGING

(71) Applicant: OPTOSURGICAL LLC, Columbia, MD (US)

(72) Inventors: De Ru Tsai, Columbia, MD (US); Yoseph Kim, Columbia, MD (US)

(21) Appl. No.: 19/556,346

(22) Filed: Mar. 4, 2026

Related U.S. Application Data
(60) Provisional application No. 63/766,514, filed on Mar. 4, 2025.

(51) Int. Cl. A61B 1/313; A61B 1/00; A61B 1/05; A61B 34/30

(52) U.S. Cl. CPC A61B 1/3132; A61B 1/00004; A61B 1/0005; A61B 1/0008; A61B 1/00181; A61B 1/00194; A61B 1/05; A61B 34/30; A61B 2034/301

(57) ABSTRACT

A system for generating a multi-angle visualization of tissue of a patient. The system includes a medical device having one or more imaging devices disposed at the distal end for measuring a continuous stream of 2D quantitative visual data. The system further includes a computing device configured to accept the one or more continuous streams of 2D quantitative visual data from the one or more imaging devices, generate a depth map, backproject the depth map into 3D object space, resulting in a 3D point cloud, define a virtual camera position relative to the 3D point cloud, generate, based on the 3D point cloud and the virtual camera position, the multi-angle visualization of the tissue, and display the multi-angle visualization of the tissue.

### CROSS-REFERENCE TO RELATED APPLICATIONS

This application is a non-provisional and claims benefit of U.S. Provisional Application No. 63/766,514 filed Mar. 4, 2025, the specification of which is incorporated herein in its entirety by reference.

### STATEMENT REGARDING FEDERALLY SPONSORED RESEARCH OR DEVELOPMENT

This invention was made with government support under Contract No. 75N91023C00048 awarded by the Advanced Research Projects Agency for Health (ARPA-H), the National Institutes of Health, and the Department of Health and Human Services. The government has certain rights in the invention.

### FIELD OF THE INVENTION

The present invention is directed to algorithms and medical devices for generating multi-angle view images of tissues for interactive visualization.

### BACKGROUND OF THE INVENTION

For medical procedures, it is of great use for devices used in these procedures to have comprehensive imaging capabilities. This allows for the medical practitioners carrying out these procedures to have the visual information required to strategize and execute said procedure safely and accurately. This is applicable to a wide variety of medical procedures that affect both internal and external tissue.

For example, laparoscopy is a minimally invasive procedure for visualizing tissue. This procedure implements a tool called a laparoscope, a thin, telescopic rod with a video camera on the end. The surgeon directs the laparoscope through a small incision in the abdomen, measuring half an inch or less. The laparoscope camera projects an image of the inside of your belly or pelvis onto a monitor in real-time. Using these images, surgeons can watch their hand motions during interactions with tissue.

In another example, imaging devices can be used to image a patient's external tissue (e.g., body surface) in order to determine surgical markings, visualize internal organs, etc. This can be carried out through motorized arms with cameras disposed thereon to allow for steady capture of visual data. These motorized arms can be controlled either manually by a medical practitioner, or by an automated program. This can allow for the medical practitioners to prepare a detailed plan for the procedure that is personalized to the particular patient.

It is advantageous for these devices to provide multiple views of tissue during use. This is generally achieved by the use of multiple imaging devices disposed around the tip of the device. The views captured by these imaging devices are combined to generate a wider field of view than could be captured with only one imaging device. Alternatively, each imaging device could capture its own separate view of the tissue of the patient, allowing the surgeon to visualize these different views simultaneously. These systems enhance surgeon perception by providing binocular depth cues while maintaining established laparoscopic workflows, including monitor-based viewing, conventional camera handling, and standard accessory use. Despite these advances, many current systems continue to rely primarily on forward-facing visualization and require manual camera repositioning to obtain alternative viewpoints. As with other prior visualization platforms, improvements in visualization are typically achieved through incremental refinements in optics, camera configuration, and video processing, rather than changes to surgical intent or technique.

Predicate devices utilize stereoscopic imaging and video processing to generate 3D visualization for minimally invasive procedures. These systems acquire video signals from laparoscopic cameras and process them to display stereoscopic images on surgical monitors, improving depth perception relative to conventional 2D systems. Surgeons interact with these systems using established controls and accessories, including monitors, camera heads, and foot pedals, consistent with standard laparoscopic practice. Clinical studies have shown that 3D laparoscopic visualization can improve depth perception and hand-eye coordination compared with 2D imaging, while maintaining comparable procedural workflows. However, similar to other prior systems, visualization is typically limited to a primary forward-facing view, and additional viewpoints require manual adjustment or repositioning of the laparoscope.

Several prior laparoscopic systems provide surgeons with adjustable viewing angles to support intraoperative visualization, including manually adjustable angled laparoscopes and multi-lens systems. These devices allow changes in viewing direction through mechanical articulation, scope rotation, or manual repositioning, while maintaining conventional laparoscopic access and visualization workflows. Although effective, manual adjustment of viewing angles may require repeated scope manipulation or repositioning, which can interrupt procedural flow. Nevertheless, these systems remain widely used and clinically accepted, as they provide additional visualization options without altering surgical intent, anatomy interaction, or procedural steps.

Overall, prior systems exist that generate 3D models of a surgical space using binocular stereo pairs of imaging devices combined with a pre-operative scan, sets of structured light imaging devices, Light Detection and Ranging (LIDAR) imaging devices, a moving image sensor, or Gaussian Splatting. However, these systems are limited to only one type of camera device (stereo), and fail to provide a complete interactive architecture to allow virtual viewpoint manipulation. Thus, there exists a present need for algorithms and medical devices capable of providing a multi-angle visualization of a patient's tissue during a surgical procedure.

### BRIEF SUMMARY OF THE INVENTION

It is an objective of the present invention to provide devices and computer-implemented methods that allow for providing a multi-angle visualization of a patient's tissue during a surgical and/or interventional procedure, as specified in the independent claims. Embodiments of the invention are given in the dependent claims. Embodiments of the present invention can be freely combined with each other if they are not mutually exclusive.

The system of the present invention receives video input from medical cameras (e.g., laparoscopic, endoscopic/capsular endoscopic, robotic, etc.) and provides real-time visualization on standard surgical monitors. The system preserves established operating room workflows and surgeon interaction paradigms while offering selectable viewing perspectives through familiar control mechanisms (e.g., foot pedal or handle-based input). These functions are consistent with the intended use and technological characteristics of existing laparoscopic video processors.

The system of the present invention provides surgeons with additional selectable viewpoints using standard monitor-based visualization. The system functions as a video processing extension that supports visualization flexibility comparable to existing multi-angle laparoscopic and/or endoscopic procedural systems. The system maintains compatibility with conventional laparoscopic and/or endoscopic equipment and preserves the established role of the surgeon/endoscopist and assistant during Minimally Invasive Surgery (MIS), endoscopic, robotic, and/or autonomous procedures.

The present invention features a system for generating a multi-angle visualization of tissue of a patient. In some embodiments, the system may comprise a medical device. The medical device may comprise a housing and one or more imaging devices disposed at the distal end of the housing, each imaging device configured to measure a continuous stream of 2D quantitative visual data. The system may further comprise a computing device configured to accept the one or more continuous streams of 2D quantitative visual data from the one or more imaging devices, generate, based on the one or more continuous streams of 2D quantitative visual data, a depth map, back-project each pixel with depth and RGB information into 3D object space, resulting in a 3D point cloud, define a virtual camera position relative to the 3D point cloud, generate, based on the 3D point cloud and the virtual camera position, the multi-angle visualization of the tissue and surgical tools along with their relative distances, and display, by a display component, the multi-angle visualization of the tissue. The system may further comprise the display component communicatively coupled to the computing device, configured to display the multi-angle visualization of the tissue and surgical tools with their relative distances to prevent from accidental damage or injury and to safely guide interventional procedures.

The present invention features a non-transitory computing medium comprising computer-readable instructions that, when executed by a processor configured to execute computer-readable instructions, configure the processor to capture, by one or more imaging devices of a medical device, one or more continuous streams of quantitative visual data. The one or more imaging devices may be disposed at a distal end of an insertable body of the medical device. The computer-readable instructions may further configure the processor to process the one or more continuous streams of quantitative visual data into 3D visual data. The computer-readable instructions may further configure the processor to generate, based on the 3D visual data, a multi-angle visualization of the tissue and surgical tools with their relative distances. The computer-readable instructions may further configure the processor to display, by a display component, the multi-angle visualization of the tissue.

One of the unique and inventive technical features of the present invention is the ability of the medical device of the present invention to measure quantitative visual data to generate a 3D point cloud. Without wishing to limit the invention to any theory or mechanism, it is believed that the technical feature of the present invention advantageously provides for real-time visualization of a multi-angle view of the patient's tissue and surgical tools with their relative distances such that a 3D real-time model can be generated and manipulated by medical officials. None of the presently known prior references or works have the unique inventive technical feature of the present invention.

Another one of the unique and inventive technical features of the present invention is the generation of a 3D point cloud with any number of angles or predetermined angled views as preset, based on a 2D monocular data stream. Without wishing to limit the invention to any theory or mechanism, it is believed that the technical feature of the present invention advantageously provides for the efficient generation of a real-time visualization of a multi-angle view of the patient's tissue and surgical tools with their relative distances or proximity without the need for multiple imaging devices or camera movement algorithms to generate an interactive visualization of the surgical space. None of the presently known prior references or works have the unique inventive technical feature of the present invention.

Furthermore, the inventive technical feature of the present invention is counterintuitive. The reason that it is counterintuitive is because it contributed to a surprising result. One of ordinary skill in the art would implement multiple camera views in order to accurately assemble multi-angle 3D visualization of a surgical space. The present invention implements a 2D monocular data stream to generate an inferred depth map which is back-projected into 3D space in order to create a dense RGB point cloud. Surprisingly, this results in an accurate multi-angle interactive 3D visualization of the surgical space based on this singular image stream. Thus, the inventive technical feature of the present invention contributed to a surprising result.

In some embodiments, the system further comprises a spatial awareness module configured to compute relative spatial relationships between anatomical structures and surgical instruments within the reconstructed 3D point cloud. The spatial awareness module generates proximity metrics, risk indicators, and safety envelopes representing spatial separation between tissues and instruments, and overlays such information onto the multi-angle visualization.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a schematic diagram of the multi-angle imaging medical device of the present invention.

FIG. 1B shows a schematic diagram of the system for multi-angle imaging of the present invention.

FIG. 1C shows a flow chart of a method for multi-angle imaging of the present invention.

FIG. 1D shows a schematic diagram of the non-transitory computing medium of the present invention for multi-angle imaging.

FIG. 2 shows a data flow chart of the multi-angle imaging process of the presently claimed invention.

FIG. 3 shows a real-time depth map generated from continuous streams of quantitative visual data by the multi-angle visualization process of the present invention.

FIG. 4 shows a real-time multi-angle view rendering generated by the process of the present invention.

FIGS. 5A-5B show examples of the user interface displayed by the multi-angle imaging system of the present invention.

FIGS. 6A-6H show various views of the 3D Multi-Angle Interactive View (3DMAV) images generated by the present invention, compared to the original 2D images captured by the imaging devices, from which the 3DMAV images were generated.

### DETAILED DESCRIPTION OF THE INVENTION

Following is a list of elements corresponding to a particular element referred to herein:
100 device
110 insertable body
112 proximal end
114 distal end
120 imaging devices
122 depth map
124 virtual camera position
126 3D point cloud
128 multi-angle visualization
130 interface component
140 spatial awareness module
200 computing device
210 processor
220 memory component
300 display component
400 robotic control system
1000 system
2000 patient
2100 tissue

The term “multi-angle view” is defined herein as a visualization that allows for a user to view the same space from multiple angles. This is distinct from a “multi-view visualization” in the sense that multi-view visualizations require the implementation of multiple imaging devices and/or image streams originating from different locations relative to the target that is being imaged.

The term “patient” is defined herein as a living or non-living organism to be imaged, encompassing both human and animal organisms.

The term “proximally” is defined herein as within 50-150 mm of the target object. This includes approaching the target object without contact, contacting the target object, or inserting into the target object.

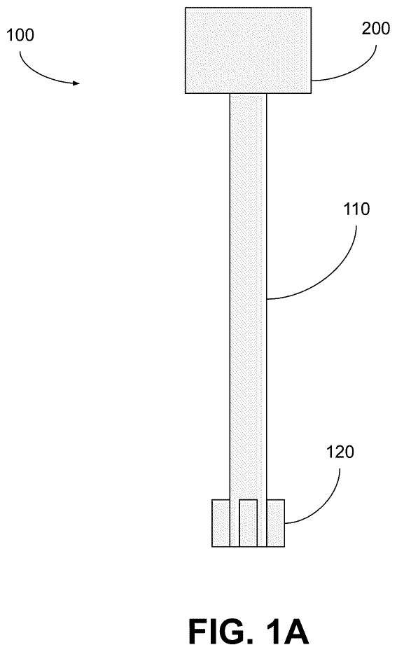

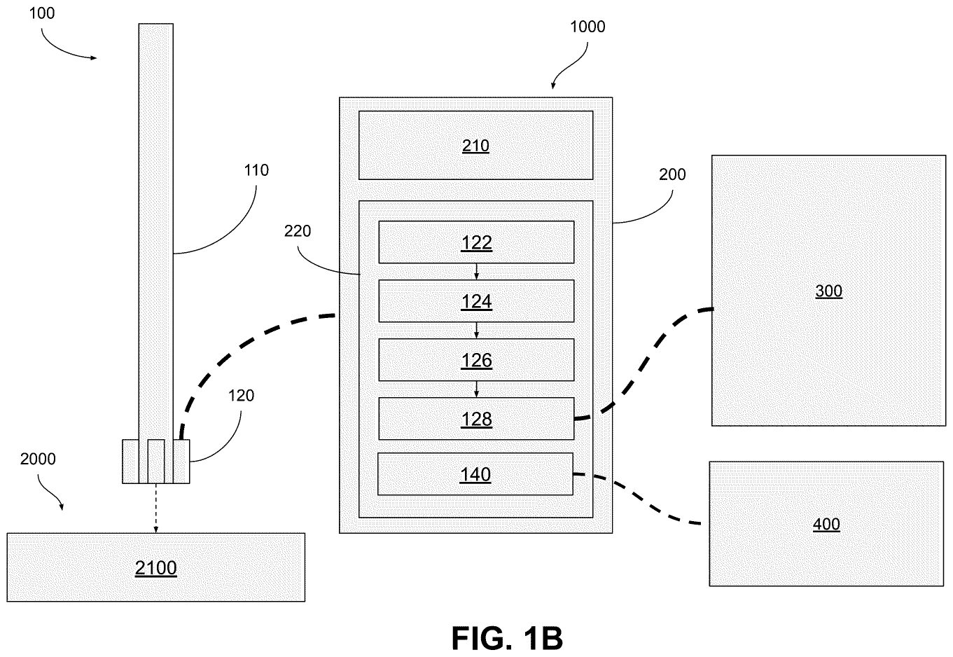

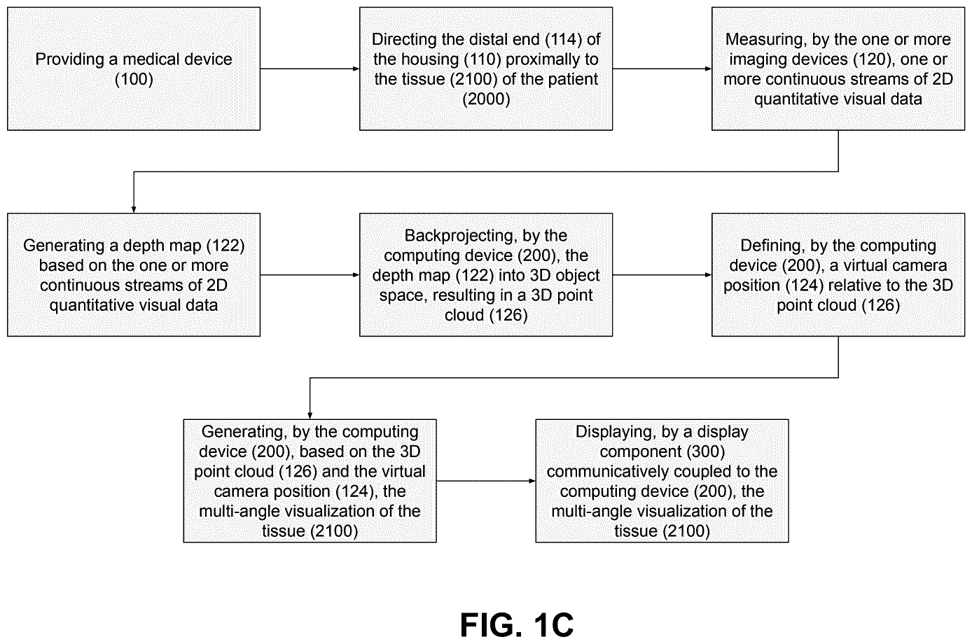

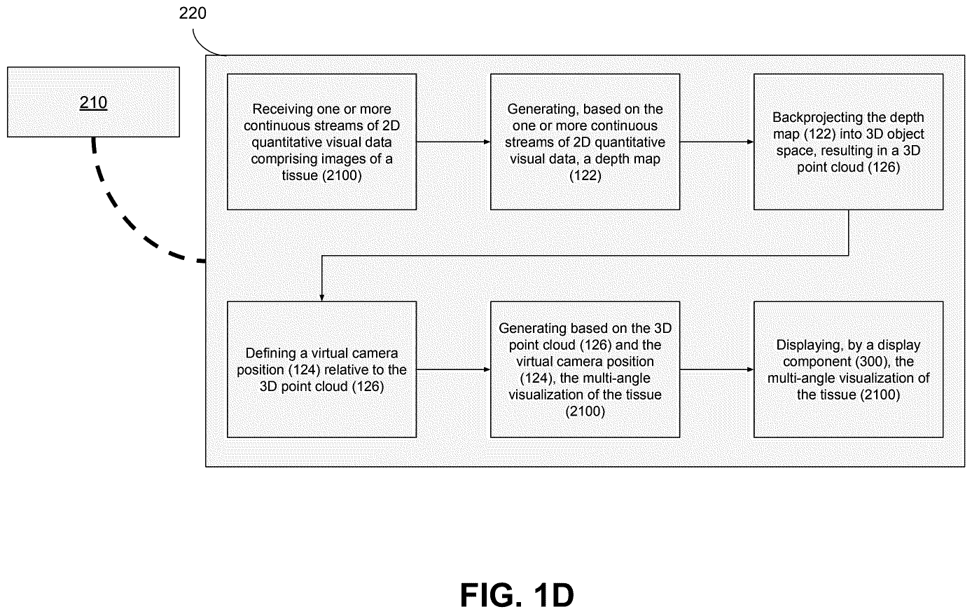

Referring now to FIG. 1B, the present invention features a system (1000) for generating a multi-angle visualization of tissue (2100) of a patient (2000). In some embodiments, the system (1000) may comprise a medical device (100). The medical device (100) may comprise a housing (110) having a proximal end (112) and a distal end (114) such that the distal end (114) of the housing (110) is configured to be directed proximally to the tissue (2100) of the patient (2000). The medical device (100) may further comprise one or more imaging devices (120) disposed at the distal end (114) of the housing (110), each imaging device configured to measure a continuous stream of 2D quantitative visual data.

The system (1000) may further comprise a computing device (200) communicatively coupled to the one or more imaging devices (120). The computing device (200) may comprise a processor (210) configured to execute computer-readable instructions, and a memory component (220) operatively coupled to the processor (210), comprising computer-readable instructions. The computer-readable instructions may comprise accepting the one or more continuous streams of 2D quantitative visual data from the one or more imaging devices (120), generating, based on the one or more continuous streams of 2D quantitative visual data, a depth map (122), backprojecting the depth map (122) into 3D object space, resulting in a 3D point cloud (126), defining a virtual camera position (124) relative to the 3D point cloud (126), generating, based on the 3D point cloud (126) and the virtual camera position (124), the multi-angle visualization of the tissue (2100), and displaying, by a display component (300), the multi-angle visualization (128) of the tissue (2100). The system (100) may further comprise the display component (300) communicatively coupled to the computing device (200), configured to display the multi-angle visualization of the tissue (2100).

In some embodiments, the one or more imaging devices (120) may comprise a single monocular camera. In some embodiments, the one or more imaging devices (120) may comprise stereo cameras, time-of-flight cameras, structured light cameras, light detection and ranging (LIDAR) cameras, or a combination thereof. In some embodiments, generating, based on the one or more continuous streams of 2D quantitative visual data, the depth map (122) may comprise a stereo matching process, a triangulation process, a time-travel-time measurement process, a 3D reconstruction process, a meshing process, a machine learning process, or a combination thereof. In some embodiments, the multi-angle visualization may comprise 8 Red-Green-Blue (RGB) images, each image comprising a different angled view of the tissue (2100). In some embodiments, the multi-angle visualization may comprise an interactive 3D point cloud (126). In some embodiments, the system (1000) may further comprise an interface component (130) configured to accept user input and adjust the multi-angle visualization based on the user input without repositioning the medical device (100). In some embodiments, the virtual camera position (124) may comprise a position simulating a position of a robotic camera arm relative to the tissue (2100).

Referring now to FIG. 1C, the present invention features a method for generating a multi-angle visualization of tissue (2100) of a patient (2000). The method may comprise providing a medical device (100). The medical device (100) may comprise a housing (110) having a proximal end (112) and a distal end (114) such that the distal end (114) of the housing (110), and one or more imaging devices (120) disposed at the distal end (114) of the housing (110). The method may further comprise directing the distal end (114) of the housing (110) proximally to the tissue (2100) of the patient (2000). The method may further comprise measuring, by the one or more imaging devices (120), one or more continuous streams of 2D quantitative visual data. The method may further comprise generating, by a computing device (200) communicatively coupled to the medical device (100), a depth map (122) based on the one or more continuous streams of 2D quantitative visual data. The method may further comprise backprojecting, by the computing device (200), the depth map (122) into 3D object space, resulting in a 3D point cloud (126). The method may further comprise defining, by the computing device (200), a virtual camera position (124) relative to the 3D point cloud (126). The method may further comprise generating, by the computing device (200), based on the 3D point cloud (126) and the virtual camera position (124), the multi-angle visualization of the tissue (2100). The method may further comprise displaying, by a display component (300) communicatively coupled to the computing device (200), the multi-angle visualization of the tissue (2100).

In some embodiments, the one or more imaging devices (120) may comprise a single monocular camera. In some embodiments, the one or more imaging devices (120) may comprise stereo cameras, time-of-flight cameras, structured light cameras, light detection and ranging (LIDAR) cameras, or a combination thereof. In some embodiments, generating, based on the one or more continuous streams of 2D quantitative visual data, the depth map (122) comprises a stereo matching process, a triangulation process, a time-travel-time measurement process, a 3D reconstruction process, a meshing process, a machine learning process, or a combination thereof. In some embodiments, the multi-angle visualization may comprise 8 Red-Green-Blue (RGB) images, each image comprising a different angled view of the tissue (2100). In some embodiments, the multi-angle visualization may comprise an interactive 3D point cloud (126). In some embodiments, the method may further comprise accepting, by an interface component (130) operatively coupled to the computing device (200), user input to adjust the multi-angle visualization without repositioning the medical device (100). In some embodiments, the virtual camera position (124) may comprise a position simulating a position of a robotic camera arm relative to the tissue (2100).

Referring now to FIG. 1D, the present invention features a non-transitory computing medium (220) comprising computer-readable instructions that, when executed by a processor (210) configured to execute the computer-readable instructions, configure the processor (210) to receive one or more continuous streams of 2D quantitative visual data comprising images of a tissue (2100). The computer-readable instructions may further configure the processor (210) to generate, based on the one or more continuous streams of 2D quantitative visual data, a depth map (122). The computer-readable instructions may further configure the processor (210) to backproject, by the computing device (200), the depth map (122) into 3D object space, resulting in a 3D point cloud (126). The computer-readable instructions may further configure the processor (210) to define a virtual camera position (124) relative to the 3D point cloud (126). The computer-readable instructions may further configure the processor (210) to generate, based on the 3D point cloud (126) and the virtual camera position (124), the multi-angle visualization of the tissue (2100). The computer-readable instructions may further configure the processor (210) to display, by a display component (300), the multi-angle visualization of the tissue (2100).

In some embodiments, the one or more continuous streams of 2D quantitative visual data may originate from a single monocular camera. In some embodiments, the multi-angle visualization may comprise an interactive 3D point cloud (126). In some embodiments, the computer-readable instructions may further comprise accepting, by an interface component (130), user input to adjust the multi-angle visualization without repositioning the medical device (100).

The present invention features a medical device (100) for generating a multi-angle visualization of tissue (2100) of a patient (2000). In some embodiments, the device (100) may comprise an insertable body (110) having a proximal end (112) and a distal end (114) such that the distal end (114) of the insertable body (110) is configured to be directed into the tissue (2100) of the patient (2000). The device (100) may further comprise one or more imaging devices (120) disposed at the distal end (114) of the insertable body (110), each imaging device configured to measure a continuous stream of quantitative visual data. The device (100) may further comprise a processing unit (200) communicatively coupled to the one or more imaging devices (120), configured to accept the one or more continuous streams of quantitative visual data from the one or more imaging devices (120), process the one or more continuous streams of quantitative visual data into 3D visual data, and generate, based on the 3D visual data, the multi-angle visualization of the tissue (2100).

In some embodiments, the one or more imaging devices (120) may comprise stereo cameras, time-of-flight cameras, structured light cameras, light detection and ranging (LIDAR) cameras, or a combination thereof. In some embodiments, processing the one or more continuous streams of quantitative visual data into the 3D visual data may comprise a stereo matching process, a triangulation process, a time-travel-time measurement process, a 3D reconstruction process, a meshing process, a machine learning process, or a combination thereof. In some embodiments, the 3D visual data may comprise one or more depth maps, one or more 3D point clouds, one or more meshes, or a combination thereof. In some embodiments, generating the multi-angle visualization of the tissue (2100) based on the 3D visual data may comprise a depth image-based rendering process, a reprojection process, a Gaussian splatting process, a screen-space interpolation process, a mesh rendering process, a machine learning process, or a combination thereof. In some embodiments, the multi-angle visualization may comprise a plurality of Red-Green-Blue (RGB) images, an interactive 3D point cloud, or a combination thereof.

The present invention features a system (1000) for generating a multi-angle visualization of tissue (2100) of a patient (2000). In some embodiments, the system (1000) may comprise a medical device (100). The device (100) may comprise an insertable body (110) having a proximal end (112) and a distal end (114) such that the distal end (114) of the insertable body (110) is configured to be directed into the tissue (2100) of the patient (2000). The device (100) may further comprise one or more imaging devices (120) disposed at the distal end (114) of the insertable body (110), each imaging device configured to measure a continuous stream of quantitative visual data.

The system (1000) may further comprise a computing device (200) communicatively coupled to the one or more imaging devices (120). The computing device (200) may comprise a processor (210) configured to execute computer-readable instructions. The computing device (200) may further comprise a memory component (220) operatively coupled to the processor (210), comprising computer-readable instructions. The computer-readable instructions may comprise accepting the one or more continuous streams of quantitative visual data from the one or more imaging devices (120). The computer-readable instructions may further comprise processing the one or more continuous streams of quantitative visual data into 3D visual data. The computer-readable instructions may further comprise generating, based on the 3D visual data, the multi-angle visualization of the tissue (2100). The computer-readable instructions may further comprise displaying, by a display component (300), the multi-angle visualization of the tissue (2100). The system (1000) may further comprise the display component (300) communicatively coupled to the computing device (200).

In some embodiments, the display component (300) may display the multi-angle visualization of the tissue (2100) such that multiple angles of the tissue (2100) are displayed concurrently (e.g. a grid formation). In some embodiments, the display component (300) may display the multi-angle visualization of the tissue (2100) such that one angle of the multiple angles is shown at a time. The display component (300) may cycle through each angle of the multiple angles upon user input. In some embodiments, the multiple angles provided by the multi-angle visualization may comprise 2 to 20 different angles.

In some embodiments, the one or more imaging devices (120) may comprise stereo cameras, time-of-flight cameras, structured light cameras, light detection and ranging (LIDAR) cameras, or a combination thereof. In some embodiments, processing the one or more continuous streams of quantitative visual data into the 3D visual data may comprise a stereo matching process, a triangulation process, a time-travel-time measurement process, a 3D reconstruction process, a meshing process, a machine learning process, or a combination thereof. In some embodiments, the 3D visual data may comprise one or more depth maps, one or more 3D point clouds, one or more meshes, or a combination thereof. In some embodiments, generating the multi-angle visualization of the tissue (2100) based on the 3D visual data may comprise a depth image-based rendering process, a reprojection process, a Gaussian splatting process, a screen-space interpolation process, a mesh rendering process, a machine learning process, or a combination thereof. In some embodiments, the multi-angle visualization may comprise a plurality of Red-Green-Blue (RGB) images. In some embodiments, the multi-angle visualization may comprise an interactive 3D point cloud. In some embodiments, the system (1000) may further comprise an interface component (130) configured to accept user input and manipulate the multi-angle visualization based on the user input.

The present invention features a method for generating a multi-angle visualization of tissue (2100) of a patient (2000). In some embodiments, the method may comprise providing a medical device (100). The device (100) may comprise an insertable body (110) having a proximal end (112) and a distal end (114). The device (100) may further comprise one or more imaging devices (120) disposed at the distal end (114) of the insertable body (110). The method may further comprise directing the distal end (114) of the insertable body (110) into the tissue (2100) of the patient (2000). The method may further comprise capturing, by the one or more imaging devices (120), one or more continuous streams of quantitative visual data. The method may further comprise processing, by a processing unit (200), the one or more continuous streams of quantitative visual data into 3D visual data. The method may further comprise generating, based on the 3D visual data, the multi-angle visualization of the tissue (2100).

In some embodiments, the one or more imaging devices (120) may comprise stereo cameras, time-of-flight cameras, structured light cameras, light detection and ranging (LIDAR) cameras, or a combination thereof. In some embodiments, processing the one or more continuous streams of quantitative visual data into the 3D visual data may comprise a stereo matching process, a triangulation process, a time-travel-time measurement process, a 3D reconstruction process, a meshing process, a machine learning process, or a combination thereof. In some embodiments, the 3D visual data may comprise one or more depth maps, one or more 3D point clouds, one or more meshes, or a combination thereof. In some embodiments, generating the multi-angle visualization of the tissue (2100) based on the 3D visual data may comprise a depth image-based rendering process, a reprojection process, a Gaussian splatting process, a screen-space interpolation process, a mesh rendering process, a machine learning process, or a combination thereof. In some embodiments, the multi-angle visualization may comprise a plurality of Red-Green-Blue (RGB) images, an interactive 3D point cloud, or a combination thereof.

Referring now to FIG. 1D, the present invention features a non-transitory computing medium (220) comprising computer-readable instructions that, when executed by a processor (210) configured to execute computer-readable instructions, configure the processor (210) to capture, by one or more imaging devices (120) of a medical device (100), one or more continuous streams of quantitative visual data. The one or more imaging devices (120) may be disposed at a distal end (114) of an insertable body (110) of the medical device (100). The computer-readable instructions may further configure the processor (210) to process the one or more continuous streams of quantitative visual data into 3D visual data. The computer-readable instructions may further configure the processor (210) to generate, based on the 3D visual data, a multi-angle visualization of the tissue (2100). The computer-readable instructions may further configure the processor (210) to display, by a display component (300), the multi-angle visualization of the tissue (2100).

In some embodiments, the one or more imaging devices (120) may comprise stereo cameras, time-of-flight cameras, structured light cameras, light detection and ranging (LIDAR) cameras, or a combination thereof. In some embodiments, processing the one or more continuous streams of quantitative visual data into the 3D visual data may comprise a stereo matching process, a triangulation process, a time-travel-time measurement process, a 3D reconstruction process, a meshing process, a machine learning process, or a combination thereof. In some embodiments, the 3D visual data may comprise one or more depth maps, one or more 3D point clouds, one or more meshes, or a combination thereof. In some embodiments, generating the multi-angle visualization of the tissue (2100) based on the 3D visual data may comprise a depth image-based rendering process, a reprojection process, a Gaussian splatting process, a screen-space interpolation process, a mesh rendering process, a machine learning process, or a combination thereof. In some embodiments, the multi-angle visualization may comprise a plurality of Red-Green-Blue (RGB) images, an interactive 3D point cloud, or a combination thereof.

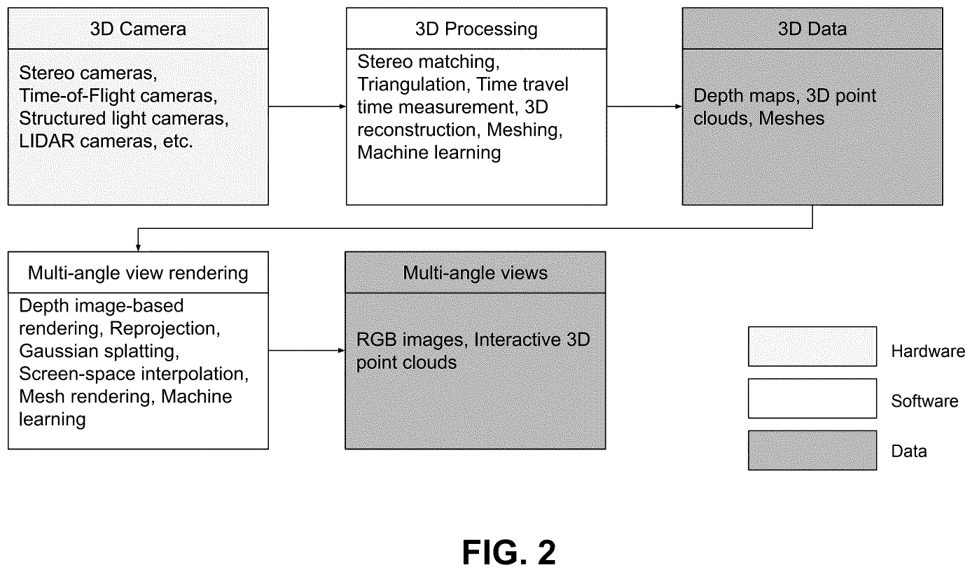

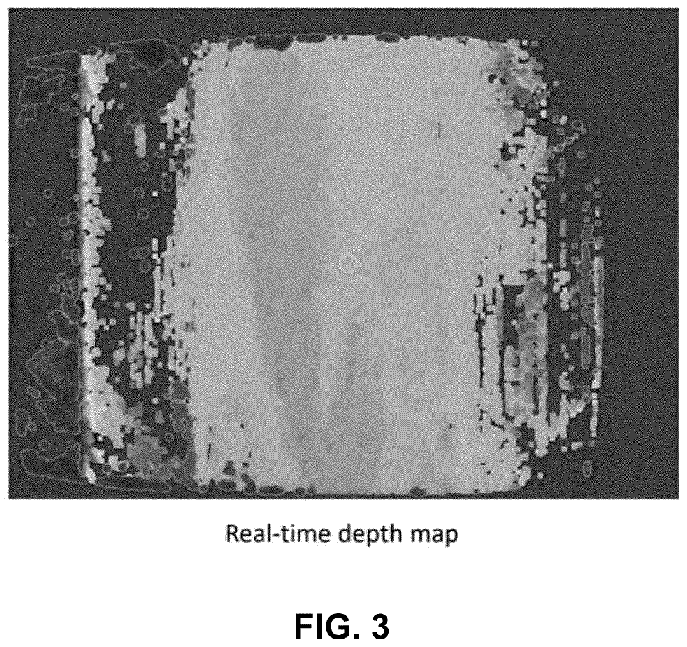

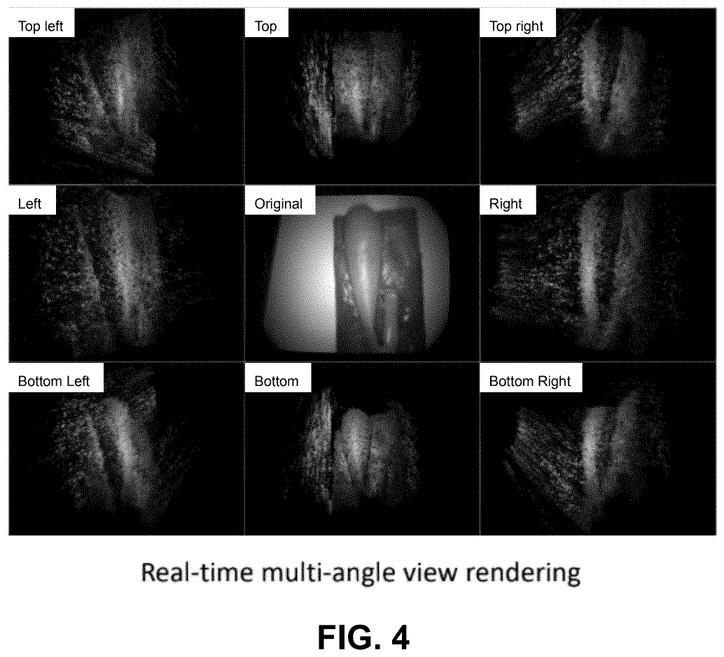

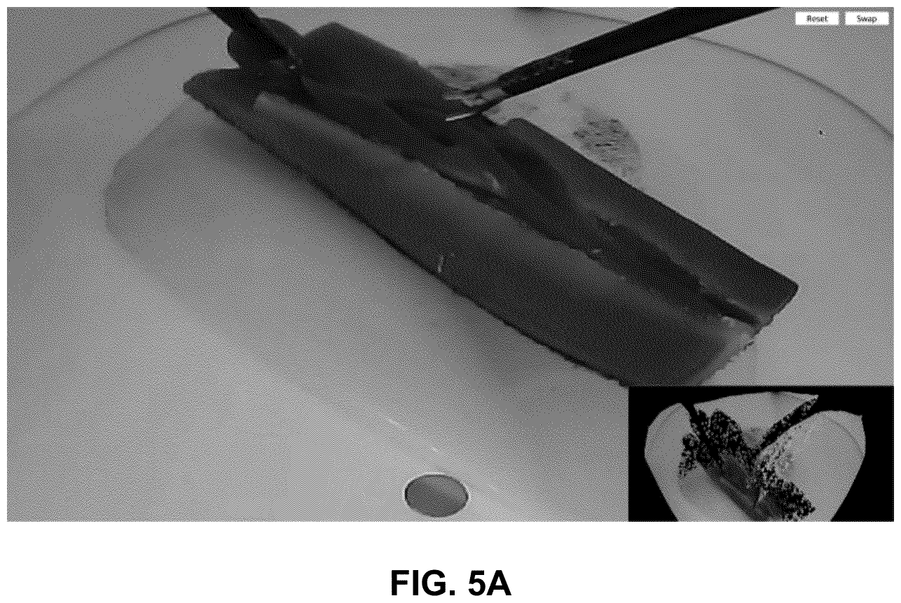

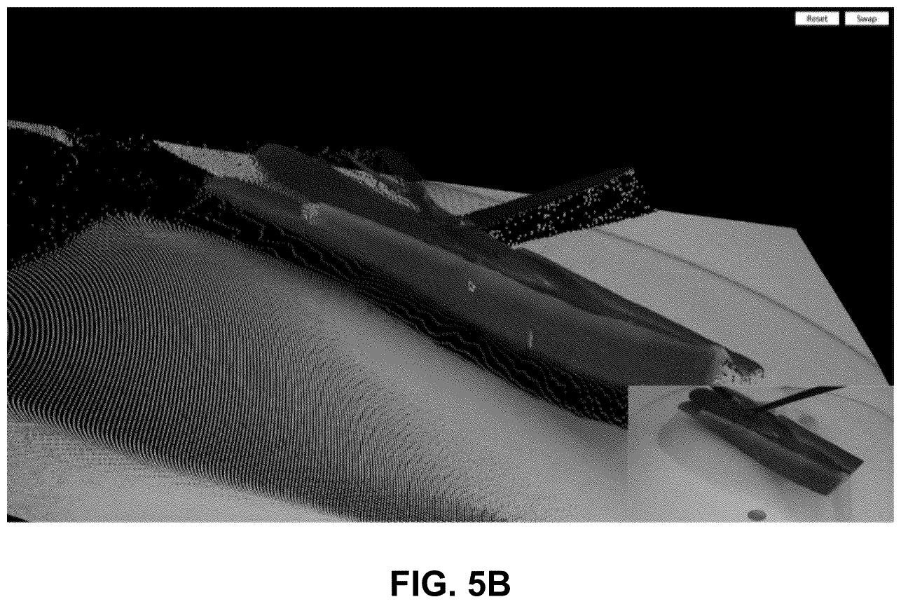

Referring now to FIG. 2, the present invention features a combination of hardware, software, and data flow to generate multi-angle views of patient tissue. The hardware may comprise 3D medical cameras selected from a group comprising stereo cameras, time-of-flight cameras, structured light cameras, and LIDAR cameras. In some embodiments, the one or more imaging devices comprise one or more cameras embedded within a capsule endoscope. The system may operate independently of imaging modality, including monocular, stereo, multispectral, hyperspectral, fluorescence, ultrasound, or hybrid imaging. The capsule endoscope may include forward-facing, side-facing, rear-facing, or circumferential imaging sensors configured to capture continuous or intermittent 2D visual data during passive or active locomotion through the gastrointestinal tract. The software may comprise 3D processing software configured to process the output from the cameras into 3D data. The 3D processing software may comprise stereo matching software, triangulation software, time-travel-time measurement software, 3D reconstruction software, meshing software, machine learning software, or a combination thereof. The 3D data may comprise depth maps, 3D point clouds, meshes, or a combination thereof. An example of a generated depth map is depicted in FIG. 3. The software may further comprise multi-angle view rendering software configured to process the 3D data into multi-angle views. The multi-angle view rendering software may comprise depth image-based rendering software, reprojection software, Gaussian splatting software, screen-space interpolation software, mesh rendering software, machine learning software, or a combination thereof. The multi-angle views may comprise RGB images, interactive 3D point clouds, or a combination thereof. An example of an interactive 3D point cloud rendering is depicted in FIG. 4.

In some embodiments, the memory component (220) may further comprise a spatial awareness module (140) comprising computer-readable instructions for estimating proximity information comprising a relative distance between one or more surgical instruments and the tissue (2100) within the 3D point cloud (126). In some embodiments, the spatial awareness module (140) may further comprise computer-readable instructions for generating proximity alerts when the relative distance between the one or more surgical instruments and the tissue (2100) falls below a threshold. In some embodiments, the multi-angle visualization (128) may further comprise a dynamic overlay displaying the proximity information. In some embodiments, the spatial awareness module (140) may further comprise computer-readable instructions for communicating the proximity information to a robotic control system (400). In some embodiments, the robotic control system (400) may be configured to modify motion trajectories based on proximity information derived from the 3D point cloud. In some embodiments, generating the 3D point cloud (126) may comprise a temporal fusion of depth maps across sequential frames.

In some embodiments, the medical device of the present invention may be an insertable device, such as a laparoscope, an endoscope, a capsule endoscope, or any other insertable medical device. In some embodiments, the medical device comprises a capsule endoscopy device configured to be ingested by a patient and to traverse a gastrointestinal lumen while capturing image data. In capsule endoscopy embodiments, the computing device reconstructs a 3D representation of luminal anatomy from sequential monocular image frames acquired during capsule motion. Temporal motion of the capsule is leveraged to infer depth and spatial relationships through structure-from-motion, optical flow, or machine-learning-based depth estimation techniques.

In some embodiments, the medical device of the present invention may be a non-insertable device, such as an optical coherence tomography device, an ultrasound imaging device, a magnetic resonance imaging (MRI) device, or any other non-insertable medical device. In some embodiments, the medical device may be controlled by a motorized mechanism (e.g., a motorized arm), operated either manually through user input, autonomously through a predetermined program, or a combination thereof.

The system of the present invention is an external, standalone video processor that receives real-time 2D laparoscopic video signals (HDMI/DVI/SDI) from imaging devices and applies GPU-accelerated image-processing algorithms to generate 3D-angle transformations. The system outputs enhanced 2D multi-angle visualization signals to standard operating room monitors. Imaging modes and viewing angles can be adjusted by the operator through an optional foot-pedal control or a handle device.

The system of the present invention may be configured to receive a video input (e.g., an HDMI/SDI video input). The video input may be compatible with existing medical imaging devices (e.g., laparoscope devices) with signal integrity across different cable lengths and routing configurations. The system may additionally be configured to implement safe pass-through behavior in case of system failure or a reboot, and processes for handling variations in color profiles and exposure/illumination changes.

The system of the present invention may be configured to generate a video output (e.g., an HDMI/SDI video input). The video output may have display compatibility across diverse monitor types, such as those implemented in an Operating Room (OR) (e.g., HD, 4k, medical-grade). The system may generate the video output in a consistent format regardless of input source variability. Furthermore, the system may implement a fail-safe mode that reverts the video output to a stable pass-through if the system is not operational.

The system of the present invention may comprise one or more accessory devices for controlling a virtual camera to navigate the 3D visualization present in the video output. In some embodiments, the virtual camera position is decoupled from the physical pose of the capsule endoscope and may be repositioned to simulate viewpoints external to the capsule, including side-angled, oblique, or retrograde views of gastrointestinal tissue not directly visible in the raw capsule video. The one or more accessory devices may comprise a wired analog input, a wireless input communicatively coupled to the system by a wireless connection (e.g., Bluetooth Low Energy, RF, etc.), or a combination thereof.

The architecture of the present system is described as follows. An incoming 2D laparoscopic video signal is first sent to a Signal Splitter, which forwards a video signal stream to the Pre-processing Module. The pre-processed video signal is then passed to the Post-processing Module, which outputs a depth map. The depth map is fed into the Rendering Module, which produces the final enhanced 2D multi-angle visualization signals to standard operating room monitors. An Interactive UI receives user commands and provides user settings to the Pre-processing, Post-processing, and Rendering Modules through a shared control line, allowing the user to adjust processing parameters and visualization options in real time. User commands include pre-processing algorithm options, post-processing algorithm options, and rendering settings (left/right/up/down/zoom-in/zoom-out/brightness/contrast/saturation).

The systems and methods of the present invention comprise a GPU-accelerated depth-estimation model to reconstruct a real-time, interactive point cloud from a standard laparoscopic video stream. This technology enhances spatial awareness beyond traditional single-view imaging. The model infers depth by analyzing geometric and semantic cues, including illumination, shading, and other factors that influence the appearance of the scene. The resulting depth map is back-projected into 3D space to create a dense RGB point cloud. The system uses virtual camera intrinsics for this back-projection because the 3D rendering is intended solely for qualitative visualization of relative spatial relationships. The visualization provides relative, probabilistic, and inferred spatial relationships rather than absolute measurements, and therefore does not require calibrated intrinsics. User inputs, including rotation angle, zoom-in, and zoom-out, are converted into a virtual camera pose and field of view, which are then applied to the point cloud to generate a new viewpoint. The transformed 3D points are projected onto the image plane and combined with a depth-aware visibility check to resolve occlusions. This process produces a re-rendered image that updates in real time as the user interacts and manipulates the view.

The systems and methods of the present invention provide two primary on-screen views: an Original View, which displays the unprocessed laparoscopic video and mirrors the standard laparoscopic monitor, and an Enhanced View, which shows a depth-based, side-angled reconstruction derived from the same live signal. In some embodiments, the Original View remains the baseline for navigation, while the Enhanced View provides additional depth and spatial context.

The user interface of the present invention is described as follows. User interaction is performed through a small set of hardware buttons and arrow controls. The Change button toggles the display between the Original View and the Enhanced View. Three arrow button pairs allow the user to adjust the virtual camera without repositioning the medical device: Arrow pair #1 (left/right) rotates the view around the X-axis in ±10° increments per click, Arrow pair #2 (up/down) rotates around the Y-axis in ±10° increments per click, and Arrow pair #3 (zoom in/out) performs digital zoom in ±1-level steps. User interaction with the visualization interface modifies the virtual camera pose, field of view, and rendering parameters, causing real-time recomputation of projected point clouds and multi-angle views.

The system of the present invention comprises a video processor unit (VPU). The architecture includes a Signal Splitter to receive a real-time video signal and forward it to a Pre-processing Module for 3D transformation. This configuration enables the system to provide a zero-latency pass-through of the original view while simultaneously reconstructing an interactive point cloud. The Signal Splitter is configured to convert 2D RGB image streams into 3D spatial representations through a multi-stage pipeline comprising: (i) semantic segmentation of anatomical structures and instruments, (ii) monocular or multi-view depth inference, (iii) temporal depth refinement across sequential frames, (iv) point cloud reconstruction via back-projection, (v) surface reconstruction or volumetric modeling, and (vi) virtual camera reprojection for multi-angle visualization.

The Pre-processing Module may be configured to resize the frame to the depth estimation model's input size while preserving the aspect ratio. The Pre-processing Module may be further configured to center-crop or letterbox the frame to match the target aspect ratio without stretching. The Pre-processing Module may be further configured to convert the frame to the required format. The Pre-processing Module may be further configured to normalize the frame using the scaling and mean/std to make it compatible with the depth estimation model. The Pre-processing Module may be further configured to apply white balance and color correction to improve appearance consistency. The Pre-processing Module may be further configured to optionally undistort or rectify the image to correct lens geometry if necessary. The Pre-processing Module may be further configured to optionally apply a mask or segmentation step to focus on the region of interest and suppress the background.

The system accepts one or more continuous streams of visual data from a variety of imaging modalities, including stereo, time-of-flight, structured light, and LIDAR cameras. The User Interface Module allows for mode selection between monocular and binocular input, ensuring compatibility with both legacy 2D systems and advanced 3D camera arrays. This User Interface Module allows for the system to accommodate multiple different imaging types at a time. The system performs real-time 2D-to-3D multi-angle transformations regardless of the input sensor type.

The system utilizes a dedicated Graphics Processing Unit (GPU) processing engine and array-based parallel computing to perform real-time depth estimation and geometric synthesis. Depth estimation and geometric synthesis using GPU processing may be performed using at least one of: 1) monocular depth neural networks, 2) stereo disparity estimation, 3) optical flow-based depth inference, 4) shape-from-shading algorithms, 5) structure-from-motion techniques, 6) neural radiance field (NeRF) approximations, 7) Gaussian splatting, and 8) hybrid physics-informed neural models. The system is model-agnostic and configured to operate with interchangeable depth estimation models. Once the depth map is generated, the system back-projects the data into 3D space using virtual camera intrinsics to create a dense RGB point cloud in the camera coordinate system. By performing depth estimation on a dedicated GPU module, the system ensures the processing speed required for intraoperative interaction and visualization.

The architecture employs a shared control line to communicate user settings concurrently to the Pre-processing, Post-processing, and Rendering Modules. This synchronization using a point-cloud processing library allows for adjustment of processing parameters, such as virtual rotation or digital zoom, to translate hardware inputs into virtual camera poses, while the physical camera device remains stationary. User inputs are first converted into a virtual camera pose and field of view, and then the transformed 3D points are projected onto the virtual image planes. In some embodiments, the system concurrently renders at least eight different virtual angle views from a single visual data stream, providing a comprehensive spatial context focusing on the target. User commands are received via an interactive UI that can be controlled by hardware inputs such as foot pedals, thumb joysticks, or touchscreen interfaces. In some embodiments, the system leverages wrist kinematics to dynamically update the virtual camera pose, enabling the generation of additional views for robotic platforms.

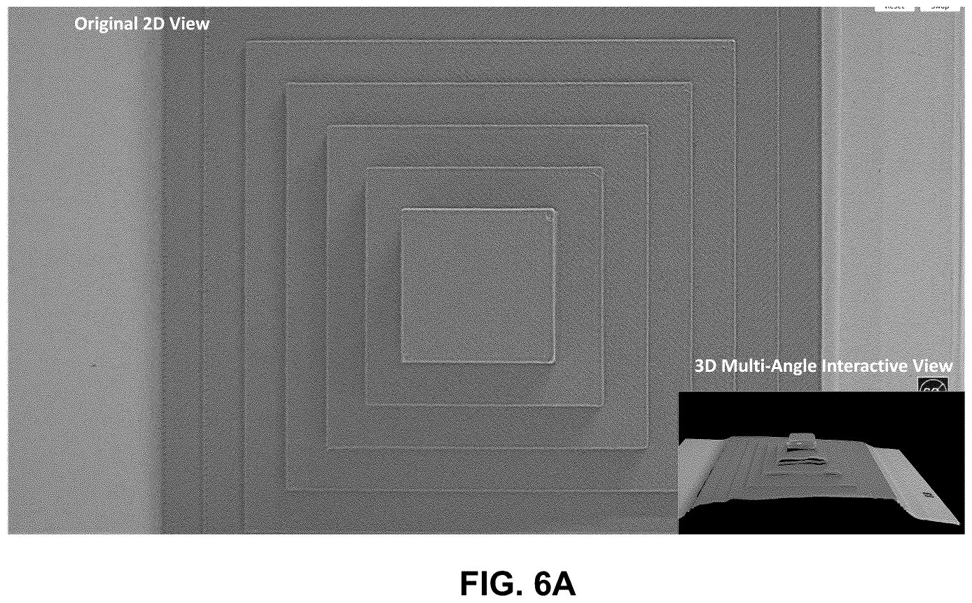

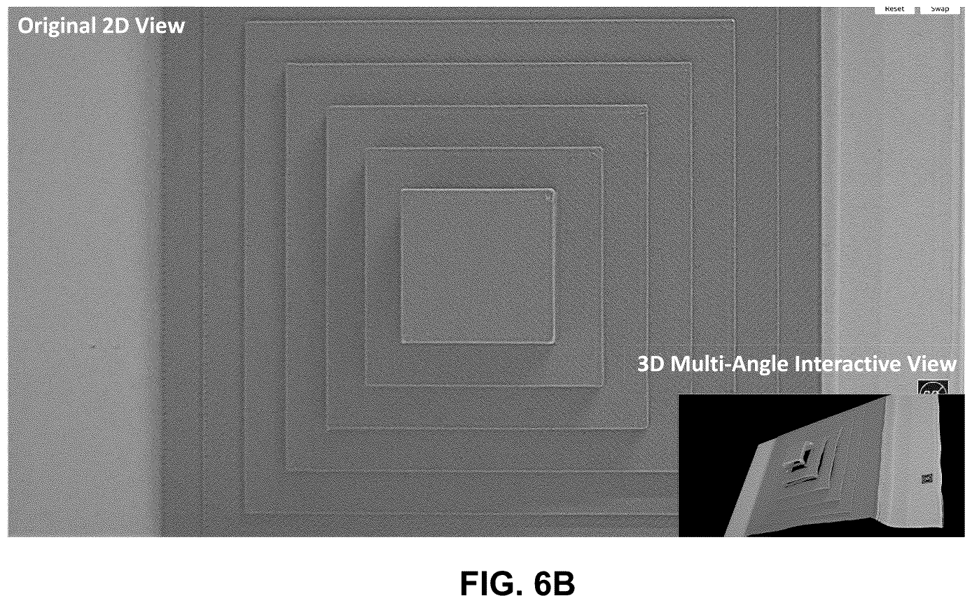

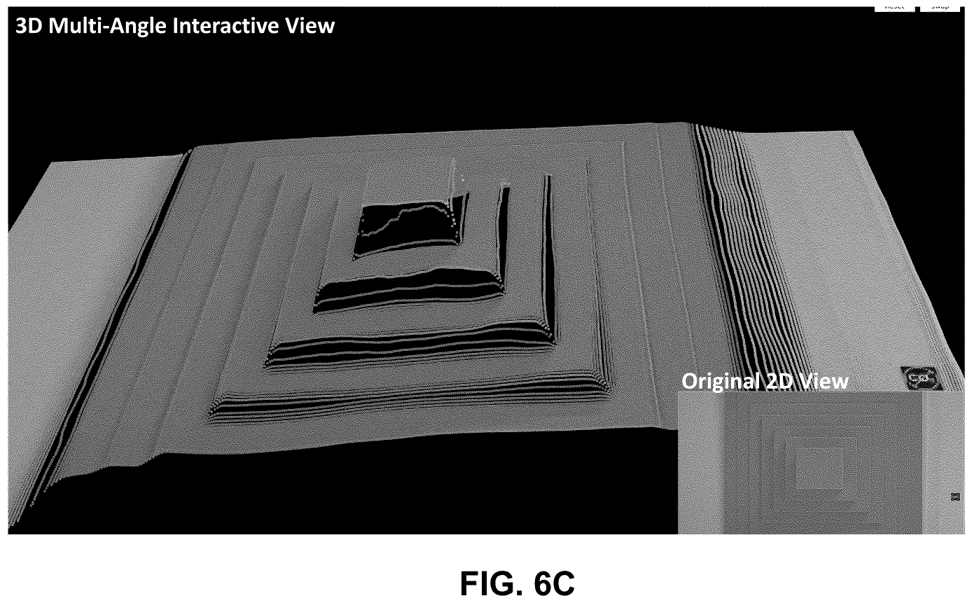

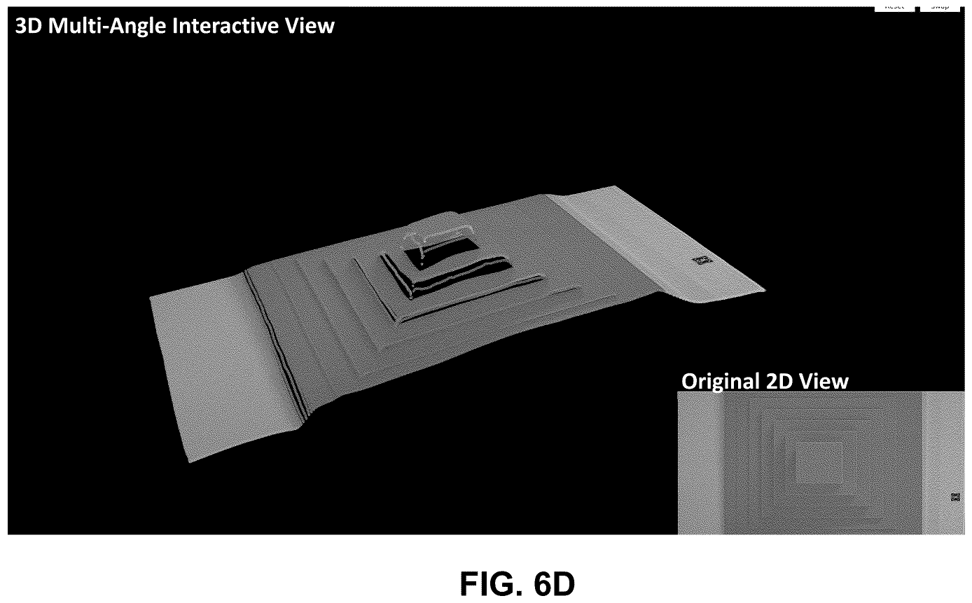

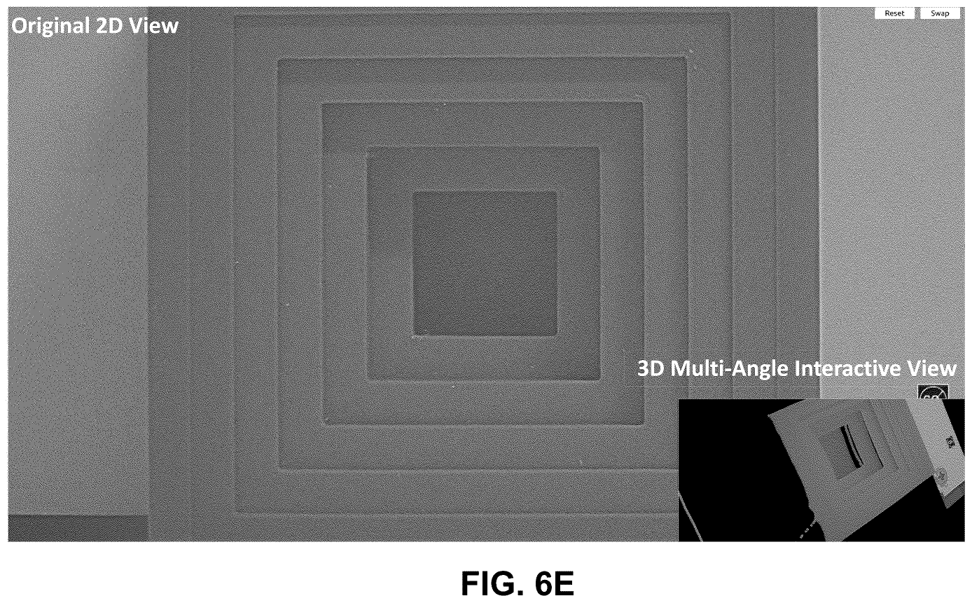

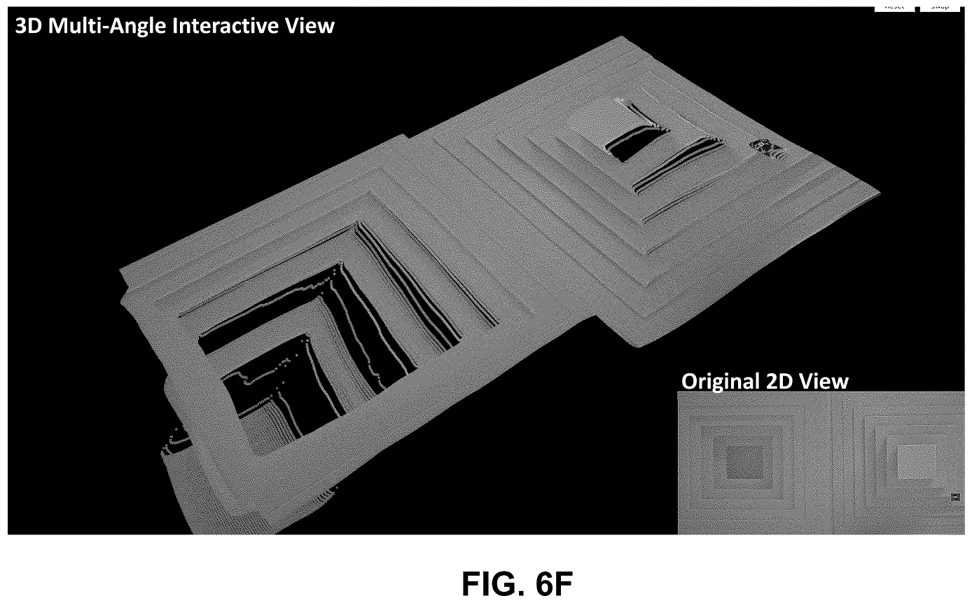

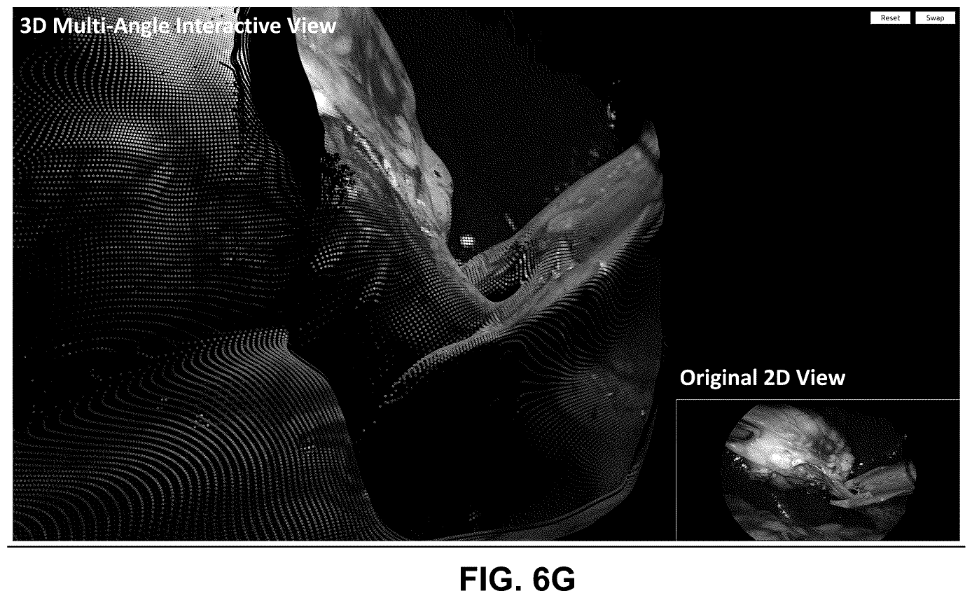

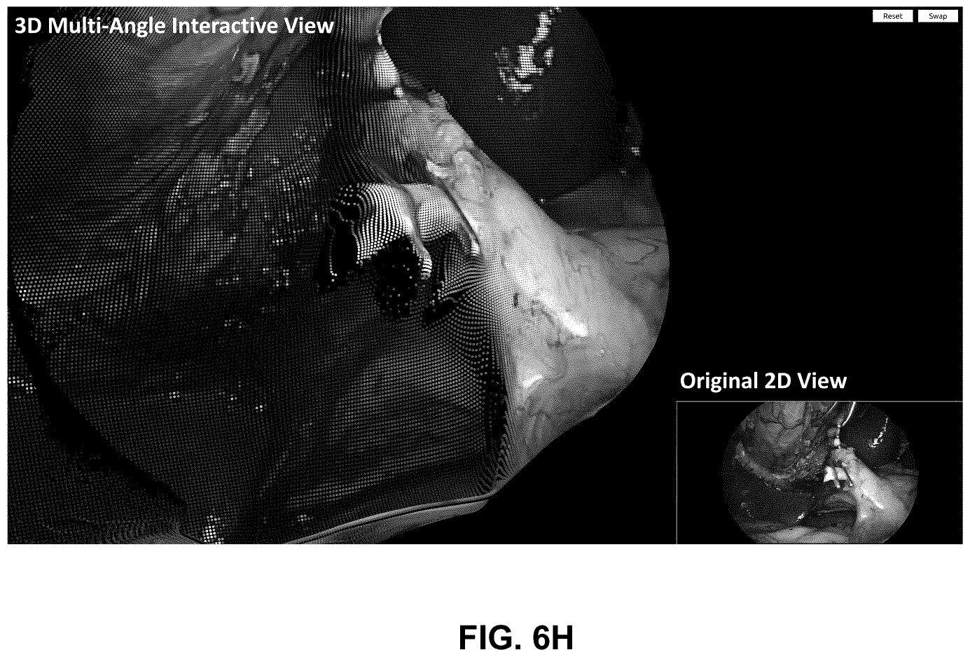

The system of the present invention is a video processor that transfers 2D video signal to 3D point cloud signal. It outputs the original video signal and 3D point cloud signal simultaneously. Imaging modes and viewing angles can be adjusted by the operator through a physical control device. The system uses ML models to reconstruct a real-time, interactive point cloud from a standard laparoscopic video stream. The model infers depth by analyzing geometric and semantic cues, including illumination, shading, and other factors that influence the appearance of the scene. The resulting depth map is back-projected into 3D space to create a dense RGB point cloud. The system uses virtual camera intrinsics for this back-projection because the 3D rendering is intended solely for qualitative visualization of relative spatial relationships. The visualization does not provide quantitative measurements, absolute positions, or surgical navigation information, and therefore does not require calibrated intrinsics. User inputs, including rotation angle, zoom-in, and zoom-out, are converted into a virtual camera pose and field of view, which are then applied to the point cloud to generate a new viewpoint. The transformed 3D points are projected onto the image plane. This process produces a re-rendered image that updates in real time as the user interacts and manipulates the view. Images of the generated interactive 3D images and their corresponding original 2D images can be seen in FIGS. 6A-6H.

In some embodiments, the virtual camera position determined by the computing device relative to the spatial data of the 3D point cloud may simulate the view captured by cameras on robotic arms with virtual cameras (e.g., wrist cameras). In some embodiments, the virtual camera position may simulate the view captured by an overhead camera, an insertable camera, or any other camera implemented in a medical setting. The resultant multi-angle visualization may resemble that captured by the medical imaging device that is being simulated.

The depth map may be generated through the use of machine learning models or algorithms. The machine learning models and/or algorithms may be specialized to the particular visual input. For example, a monocular visual input generated by a monocular camera may be fed into a monocular machine learning model trained and configured to transform the monocular visual input into a depth map. Backprojection may then be used to convert the depth map into a 3D point cloud. The 3D point cloud may be viewable as dense points in 3D space with RGB colors. A virtual camera is defined in the 3D space, and the multi-angle visualization is determined based on the relationship between the camera and the 3D point cloud. Based on the 3D point cloud, two separate visualization modes may be employed. The first mode may comprise an interactive 3D point cloud, allowing users to rotate, zoom, and pan the view of the 3D point cloud. The user may interact with the interactive 3D point cloud by an interface component, such as a keyboard, a touch screen, a set of buttons, etc. The second mode may comprise a rendering of at most 8 additional views of the 3D point cloud (top left, top, top right, left, right, bottom left, bottom, and bottom right).

In some embodiments, the system computes relative distances between objects represented in the 3D point cloud, including distances between surgical instruments and anatomical tissues, between multiple tissues, or between multiple instruments. The distances may be estimated using Euclidean distance, geodesic distance on reconstructed surfaces, or probabilistic depth uncertainty models. In some embodiments, the system may generate one or more proximity zones in the 3D point cloud, including warning zones, caution zones, and safe zones surrounding anatomical structures or instruments. The zones are dynamically updated in real time as the point cloud changes. In some embodiments, proximity information (e.g., a surgical tool approaching tissue) may be displayed using at least one of: color-coded overlays, heat maps, contour lines, volumetric bounding regions, vector arrows, numerical indicators, and auditory or haptic alerts. In some embodiments, the proximity information is provided to a robotic control system (400) or autonomous decision module to modify tool trajectories, motion constraints, or task execution based on detected spatial relationships.

In some embodiments, the system of the present invention may be compatible with any type of tissue. For example, the system of the present invention may be compatible with soft tissue, deformable tissue, static tissue, or a combination thereof. In some embodiments, the 3D visualizations of the present invention may be generated in real-time. In some embodiments, the system of the present invention may be applied directly to operating room conditions such that the imaging functionality is usable throughout a surgical/medical procedure.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R. F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., a liquid crystal display (LCD), light emitting diode (LED) display, organic light emitting diode (OLED) display, Augmented Reality (AR) display, or Virtual Reality (VR) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®. NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

The present invention may comprise or implement a neural network for machine learning tasks. These may be implemented alongside multi-angle distortion-correction algorithms. The neural network may be implemented for learning user behavior and habits, and adjusting inputs accordingly to prevent commonly-detected user errors. The neural network may be used to correct camera error (i.e., camera movement, positioning, imaging) in real-time to achieve optimal camera distance and image quality. The neural network may be used to identify optimal depths for imaging and to prioritize these depths in the generation of 3D point clouds. The neural network may be used to optimize the performance of the Computer Processing Unit, the Graphics Processing Unit, or a combination thereof.

The neural network may be stored, trained, and/or executed entirely on the user's computer, partly on the user's computer, as a stand-alone software package, partly on the user's computer and partly on a remote computer or entirely on the remote computer or server. The neural network may be stored in the form of program code, as described above. The neural network, in some embodiments, may be a perceptron neural network, a feed-forward neural network, a multilayer perceptron neural network, a convolutional neural network, a radial basis functional neural network, a recurrent neural network, a long short-term memory neural network, a sequence-to-sequence neural network model, a modular neural network, or the like.

### Embodiments

The following embodiments are intended to be illustrative only and not to be limiting in any way.
Embodiment 1: A medical imaging device (100) for generating a multi-angle visualization of tissue (2100) of a patient (2000), the device (100) comprising: a housing (110) having a proximal end (112) and a distal end (114) such that the distal end (114) of the housing (110) is configured to be directed proximally to the tissue (2100) of the patient (2000); one or more imaging devices (120) disposed at the distal end (114) of the housing (110), each imaging device configured to measure a continuous stream of quantitative visual data; and a processing unit (200) communicatively coupled to the one or more imaging devices (120), configured to accept the one or more continuous streams of quantitative visual data from the one or more imaging devices (120), process the one or more continuous streams of quantitative visual data into 3D visual data, and generate, based on the 3D visual data, the multi-angle visualization of the tissue (2100).
Embodiment 2: The device (100) of Embodiment 1, wherein the one or more imaging devices (120) comprise stereo cameras, time-of-flight cameras, structured light cameras, light detection and ranging (LIDAR) cameras, or a combination thereof.
Embodiment 3: The device (100) of Embodiment 1, wherein processing the one or more continuous streams of quantitative visual data into the 3D visual data comprises a stereo matching process, a triangulation process, a time-travel-time measurement process, a 3D reconstruction process, a meshing process, a machine learning process, or a combination thereof.
Embodiment 4: The device (100) of Embodiment 1, wherein the 3D visual data comprises one or more depth maps, one or more 3D point clouds, one or more meshes, or a combination thereof.
Embodiment 5: The device (100) of Embodiment 1, wherein generating the multi-angle visualization of the tissue (2100) based on the 3D visual data comprises a depth image-based rendering process, a reprojection process, a Gaussian splatting process, a screen-space interpolation process, a mesh rendering process, a machine learning process, or a combination thereof.
Embodiment 6: The device (100) of Embodiment 1, wherein the multi-angle visualization comprises a plurality of Red-Green-Blue (RGB) images, an interactive 3D point cloud, or a combination thereof.
Embodiment 7: A system (1000) for generating a multi-angle visualization of tissue (2100) of a patient (2000), the system (1000) comprising: a medical device (100) comprising: a housing (110) having a proximal end (112) and a distal end (114) such that the distal (114) end of the housing (110) is configured to be directed proximally to the tissue (2100) of the patient (2000); and one or more imaging devices (120) disposed at the distal end (114) of the housing (110), each imaging device configured to measure a continuous stream of quantitative visual data; a computing device (200) communicatively coupled to the one or more imaging devices (120), comprising: a processor (210) configured to execute computer-readable instructions; and a memory component (220) operatively coupled to the processor (210), comprising computer-readable instructions for: accepting the one or more continuous streams of quantitative visual data from the one or more imaging devices (120); processing the one or more continuous streams of quantitative visual data into 3D visual data; generating, based on the 3D visual data, the multi-angle visualization of the tissue (2100); and displaying, by a display component (300), the multi-angle visualization of the tissue (2100); and the display component (300) communicatively coupled to the computing device (200).
Embodiment 8: The system (1000) of Embodiment 7, wherein the one or more imaging devices (120) comprise stereo cameras, time-of-flight cameras, structured light cameras, light detection and ranging (LIDAR) cameras, or a combination thereof.
Embodiment 9: The system (1000) of Embodiment 7, wherein processing the one or more continuous streams of quantitative visual data into the 3D visual data comprises a stereo matching process, a triangulation process, a time-travel-time measurement process, a 3D reconstruction process, a meshing process, a machine learning process, or a combination thereof.
Embodiment 10: The system (1000) of Embodiment 7, wherein the 3D visual data comprises one or more depth maps, one or more 3D point clouds, one or more meshes, or a combination thereof.
Embodiment 11: The system (1000) of Embodiment 7, wherein generating the multi-angle visualization of the tissue (2100) based on the 3D visual data comprises a depth image-based rendering process, a reprojection process, a Gaussian splatting process, a screen-space interpolation process, a mesh rendering process, a machine learning process, or a combination thereof.
Embodiment 12: The system (1000) of Embodiment 7, wherein the multi-angle visualization comprises a plurality of Red-Green-Blue (RGB) images.
Embodiment 13: The system (1000) of Embodiment 7, wherein the multi-angle visualization comprises an interactive 3D point cloud.
Embodiment 14: The system (1000) of Embodiment 13 further comprising an interface component (130) configured to accept user input and manipulate the multi-angle visualization based on the user input.
Embodiment 15: A method for generating a multi-angle visualization of tissue (2100) of a patient (2000), the method comprising: providing a medical device (100) comprising: a housing (110) having a proximal end (112) and a distal end (114); and one or more imaging devices (120) disposed at the distal end (114) of the housing (110); directing the distal end (114) of the housing (110) proximally to the tissue (2100) of the patient (2000); capturing, by the one or more imaging devices (120), one or more continuous streams of quantitative visual data; processing, by a processing unit (200), the one or more continuous streams of quantitative visual data into 3D visual data; and generating, based on the 3D visual data, the multi-angle visualization of the tissue (2100).
Embodiment 16: The method of Embodiment 15, wherein the one or more imaging devices (120) comprise stereo cameras, time-of-flight cameras, structured light cameras, light detection and ranging (LIDAR) cameras, or a combination thereof.
Embodiment 17: The method of Embodiment 15, wherein processing the one or more continuous streams of quantitative visual data into the 3D visual data comprises a stereo matching process, a triangulation process, a time-travel-time measurement process, a 3D reconstruction process, a meshing process, a machine learning process, or a combination thereof.
Embodiment 18: The method of Embodiment 15, wherein the 3D visual data comprises one or more depth maps, one or more 3D point clouds, one or more meshes, or a combination thereof.
Embodiment 19: The method of Embodiment 15, wherein generating the multi-angle visualization of the tissue (2100) based on the 3D visual data comprises a depth image-based rendering process, a reprojection process, a Gaussian splatting process, a screen-space interpolation process, a mesh rendering process, a machine learning process, or a combination thereof.
Embodiment 20: The method of Embodiment 15, wherein the multi-angle visualization comprises a plurality of Red-Green-Blue (RGB) images, an interactive 3D point cloud, or a combination thereof.
Embodiment 21: A non-transitory computing medium (220) comprising computer-readable instructions that, when executed by a processor (210) configured to execute computer-readable instructions, configure the processor (210) to: capture, by one or more imaging devices (120) of a medical device (100), one or more continuous streams of quantitative visual data, wherein the one or more imaging devices (120) are disposed at a distal end (114) of a housing (110) of the medical device (100); process the one or more continuous streams of quantitative visual data into 3D visual data; generate, based on the 3D visual data, a multi-angle visualization of the tissue (2100); and display, by a display component (300), the multi-angle visualization of the tissue (2100).
Embodiment 22: The non-transitory computing medium (220) of Embodiment 21, wherein the one or more imaging devices (120) comprise stereo cameras, time-of-flight cameras, structured light cameras, light detection and ranging (LIDAR) cameras, or a combination thereof.
Embodiment 23: The non-transitory computing medium (220) of Embodiment 21, wherein processing the one or more continuous streams of quantitative visual data into the 3D visual data comprises a stereo matching process, a triangulation process, a time-travel-time measurement process, a 3D reconstruction process, a meshing process, a machine learning process, or a combination thereof.
Embodiment 24: The non-transitory computing medium (220) of Embodiment 21, wherein the 3D visual data comprises one or more depth maps, one or more 3D point clouds, one or more meshes, or a combination thereof.
Embodiment 25: The non-transitory computing medium (220) of Embodiment 21, wherein generating the multi-angle visualization of the tissue (2100) based on the 3D visual data comprises a depth image-based rendering process, a reprojection process, a Gaussian splatting process, a screen-space interpolation process, a mesh rendering process, a machine learning process, or a combination thereof.
Embodiment 26: The non-transitory computing medium (220) of Embodiment 21, wherein the multi-angle visualization comprises a plurality of Red-Green-Blue (RGB) images, an interactive 3D point cloud, or a combination thereof.
Embodiment 27: A system (1000) for generating a multi-angle visualization of tissue (2100) of a patient (2000), the system (1000) comprising: a medical device (100) comprising: a housing (110) having a proximal end (112) and a distal end (114) such that the distal end (114) of the housing (110) is configured to be directed proximally to the tissue (2100) of the patient (2000); and one or more imaging devices (120) disposed at the distal end (114) of the housing (110), each imaging device configured to measure a continuous stream of 2D quantitative visual data; a computing device (200) communicatively coupled to the one or more imaging devices (120), comprising: a processor (210) configured to execute computer-readable instructions; and a memory component (220) operatively coupled to the processor (210), comprising computer-readable instructions for: accepting the one or more continuous streams of 2D quantitative visual data from the one or more imaging devices (120); generating, based on the one or more continuous streams of 2D quantitative visual data, a depth map (122); backprojecting the depth map (122) into 3D object space, resulting in a 3D point cloud (126); defining a virtual camera position (124) relative to the 3D point cloud (126); generating, based on the 3D point cloud (126) and the virtual camera position (124), the multi-angle visualization of the tissue (2100); and displaying, by a display component (300), the multi-angle visualization (128) of the tissue (2100); and the display component (300) communicatively coupled to the computing device (200), configured to display the multi-angle visualization of the tissue (2100).
Embodiment 28: The system of Embodiment 27, further comprising a spatial awareness module configured to estimate relative distances between surgical instruments and anatomical tissues within the 3D point cloud.
Embodiment 29: The system of Embodiment 28, wherein the spatial awareness module generates proximity alerts when a distance between an instrument and tissue falls below a threshold.
Embodiment 30: The system of Embodiment 28, wherein proximity information is displayed as a dynamic overlay on the multi-angle visualization.
Embodiment 31: The system of Embodiment 27, wherein the medical device comprises a capsule endoscope configured to be ingested by a patient.
Embodiment 32: The system of Embodiment 31, wherein the one or more imaging devices are disposed within a capsule endoscope.
Embodiment 33: The system of Embodiment 31, wherein the capsule endoscope captures monocular image data while traversing a gastrointestinal lumen.
Embodiment 34: The system of Embodiment 28, wherein the spatial awareness module communicates proximity information to a robotic control system.
Embodiment 35: The system of Embodiment 34, wherein the robotic control system modifies motion trajectories based on proximity information derived from the 3D point cloud.
Embodiment 36: The system of Embodiment 27, wherein the 3D point cloud is generated from sequential monocular images acquired during motion of the capsule endoscope.
Embodiment 37: The system of Embodiment 31, wherein depth estimation for capsule endoscopy is performed using temporal image sequences rather than stereo imaging.
Embodiment 38: The system of Embodiment 27, wherein generating the 3D point cloud comprises temporal fusion of depth maps across sequential frames.
Embodiment 39: The system of Embodiment 27, wherein the system generates multi-angle views using virtual camera reprojection from arbitrary viewpoints not physically accessible by the imaging device.
Embodiment 40: The system of Embodiment 27, wherein the system supports interactive viewpoint manipulation using a user interface.
Embodiment 41: The system of Embodiment 31, wherein the multi-angle visualization provides simulated viewpoints external to the capsule endoscope.
Embodiment 42: The system of Embodiment 31, wherein the multi-angle visualization enables visualization of gastrointestinal tissue from viewpoints not physically accessible by the capsule.
Embodiment 43: The system of Embodiment 27, wherein proximity information derived from the 3D point cloud is used to assess risk of tissue contact or luminal narrowing during capsule traversal.
Embodiment 44: The system of Embodiment 27, wherein the system provides spatial awareness data to an autonomous capsule control or diagnostic module.
Embodiment 45: A method for generating a multi-angle visualization of gastrointestinal tissue, the method comprising: ingesting a capsule endoscope by a patient; capturing sequential monocular image frames during capsule traversal; generating a depth map from the image frames; reconstructing a 3D point cloud of luminal anatomy; defining one or more virtual camera viewpoints; generating a multi-angle visualization independent of capsule pose.
Embodiment 46: A non-transitory computing medium (220) comprising computer-readable instructions that, when executed by a processor (210) configured to execute the computer-readable instructions, configure the processor (210) to: receive one or more continuous streams of 2D quantitative visual data comprising images of a tissue (2100); generate, based on the one or more continuous streams of 2D quantitative visual data, a depth map (122); backproject the depth map (122) into 3D object space, resulting in a 3D point cloud (126); define a virtual camera position (124) relative to the 3D point cloud (126); generate based on the 3D point cloud (126) and the virtual camera position (124), a multi-angle visualization of the tissue (2100); and display, by a display component (300), the multi-angle visualization of the tissue (2100).
Embodiment 47: The non-transitory computing medium of Embodiment 46, wherein the image data originates from a capsule endoscope.
Embodiment 48: The non-transitory computing medium of Embodiment 46, wherein the computer-readable instructions generate 3D spatial representations from temporally displaced monocular images.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1) A system (1000) for generating a multi-angle visualization of tissue (2100) of a patient (2000), the system (1000) comprising:
a) a medical device (100) comprising:
i) a housing (110) having a proximal end (112) and a distal end (114) such that the distal end (114) of the housing (110) is configured to be directed proximally to the tissue (2100) of the patient (2000); and
ii) one or more imaging devices (120) disposed at the distal end (114) of the housing (110), each imaging device configured to measure a continuous stream of 2D quantitative visual data;
b) a computing device (200) communicatively coupled to the one or more imaging devices (120), comprising:
i) a processor (210) configured to execute computer-readable instructions; and
ii) a memory component (220) operatively coupled to the processor (210), comprising computer-readable instructions for:
A) accepting the one or more continuous streams of 2D quantitative visual data from the one or more imaging devices (120);
B) generating, based on the one or more continuous streams of 2D quantitative visual data, a depth map (122);
C) backprojecting the depth map (122) into 3D object space, resulting in a 3D point cloud (126);
D) defining a virtual camera position (124) relative to the 3D point cloud (126);
E) generating, based on the 3D point cloud (126) and the virtual camera position (124), the multi-angle visualization of the tissue (2100); and
F) displaying, by a display component (300), the multi-angle visualization (128) of the tissue (2100); and
c) the display component (300) communicatively coupled to the computing device (200), configured to display the multi-angle visualization of the tissue (2100).

2) The system (1000) of claim 1, wherein the one or more imaging devices (120) comprise a single monocular camera.

3) The system (1000) of claim 1, wherein generating, based on the one or more continuous streams of 2D quantitative visual data, the depth map (122) comprises a stereo matching process, a triangulation process, a time-travel-time measurement process, a 3D reconstruction process, a meshing process, a machine learning process, or a combination thereof.

4) The system (1000) of claim 1, wherein the multi-angle visualization comprises an interactive 3D point cloud (126).

5) The system (1000) of claim 4 further comprising an interface component (130) configured to accept user input and adjust the multi-angle visualization based on the user input without repositioning the medical device (100).

6) The system (1000) of claim 1, wherein the memory component (220) further comprises a spatial awareness module (140) comprising computer-readable instructions for estimating proximity information comprising a relative distance between one or more surgical instruments and the tissue (2100) within the 3D point cloud (126).

7) The system (1000) of claim 6, wherein the spatial awareness module (140) further comprises computer-readable instructions for generating proximity alerts when the relative distance between the one or more surgical instruments and the tissue (2100) falls below a threshold.

8) The system (1000) of claim 6, wherein the multi-angle visualization (128) further comprises a dynamic overlay displaying the proximity information.

9) The system (1000) of claim 6, wherein the spatial awareness module (140) further comprises computer-readable instructions for communicating the proximity information to a robotic control system (400).

10. The system (1000) of claim 9, wherein the robotic control system (400) is configured to modify motion trajectories based on proximity information derived from the 3D point cloud.

11. The system (1000) of claim 1, wherein generating the 3D point cloud (126) comprises a temporal fusion of depth maps across sequential frames.

12. A method for generating a multi-angle visualization of tissue (2100) of a patient (2000), the method comprising:
a) providing a medical device (100) comprising:
i) a housing (110) having a proximal end (112) and a distal end (114) such that the distal end (114) of the housing (110); and
ii) one or more imaging devices (120) disposed at the distal end (114) of the housing (110);
b) directing the distal end (114) of the housing (110) proximally to the tissue (2100) of the patient (2000);
c) measuring, by the one or more imaging devices (120), one or more continuous streams of 2D quantitative visual data;
d) generating, by a computing device (200) communicatively coupled to the medical device (100), a depth map (122) based on the one or more continuous streams of 2D quantitative visual data;
e) backprojecting, by the computing device (200), the depth map (122) into 3D object space, resulting in a 3D point cloud (126);
f) defining, by the computing device (200), a virtual camera position (124) relative to the 3D point cloud (126);
g) generating, by the computing device (200), based on the 3D point cloud (126) and the virtual camera position (124), the multi-angle visualization of the tissue (2100); and
h) displaying, by a display component (300) communicatively coupled to the computing device (200), the multi-angle visualization of the tissue (2100).

13. The method of claim 12, wherein the one or more imaging devices (120) comprise a single monocular camera.

14. The method of claim 12, wherein generating, based on the one or more continuous streams of 2D quantitative visual data, the depth map (122) comprises a stereo matching process, a triangulation process, a time-travel-time measurement process, a 3D reconstruction process, a meshing process, a machine learning process, or a combination thereof.

15. The method of claim 12, wherein the multi-angle visualization comprises an interactive 3D point cloud (126).

16. The method of claim 15 further comprising accepting, by an interface component (130) operatively coupled to the computing device (200), user input to adjust the multi-angle visualization without repositioning the medical device (100).

17. A non-transitory computing medium (220) comprising computer-readable instructions that, when executed by a processor (210) configured to execute the computer-readable instructions, configure the processor (210) to:
a) receive one or more continuous streams of 2D quantitative visual data comprising images of a tissue (2100);
b) generate, based on the one or more continuous streams of 2D quantitative visual data, a depth map (122);
c) backproject the depth map (122) into 3D object space, resulting in a 3D point cloud (126);
d) define a virtual camera position (124) relative to the 3D point cloud (126);
e) generate based on the 3D point cloud (126) and the virtual camera position (124), a multi-angle visualization of the tissue (2100); and
f) display, by a display component (300), the multi-angle visualization of the tissue (2100).

18. The non-transitory computing medium (220) of claim 17, wherein the one or more continuous streams of 2D quantitative visual data originate from a single monocular camera.

19. The non-transitory computing medium (220) of claim 17, wherein the multi-angle visualization comprises an interactive 3D point cloud (126).

20. The non-transitory computing medium (220) of claim 19, wherein the computer-readable instructions further comprise accepting, by an interface component (130), user input to adjust the multi-angle visualization without repositioning the medical device (100).
